(19) United States
(12) Patent Application Publication
(10) Pub. No.: US 2025/0251320 A1
(43) Pub. Date: Aug. 7, 2025

(54) METHODS AND SYSTEMS FOR ANALYZING IMAGES FOR PRECISION TISSUE MICROARRAY CONSTRUCTION

(71) Applicant: Noetik Inc., South San Francisco, CA (US)

(72) Inventors: Lacey Padron, Longmont, CO (US); Tyler Van Hensbergen, Portland, OR (US); Rodney Collins, San Francisco, CA (US); Jacob Rinaldi, San Francisco, CA (US); Meena Subramaniam, Fremont, CA (US); Francis Fernandez, San Francisco, CA (US); Ronald Alfa, Salt Lake City, UT (US); Hargita Kaplan, Fremont, CA (US); Kelsey Dutton, Boulder, CO (US)

(21) Appl. No.: 19/047,061

(22) Filed: Feb. 6, 2025

Related U.S. Application Data
(60) Provisional application No. 63/550,825, filed on Feb. 7, 2024.

(51) Int. Cl. G01N 1/30; G01N 1/36; G06V 10/25; G06V 10/764; G06V 20/69

(52) U.S. Cl. CPC G01N 1/30; G01N 1/36; G06V 10/25; G06V 10/764; G06V 20/698; G06V 2201/03

(57) ABSTRACT

Disclosed are systems comprising a processor; and a non-transitory computer readable medium comprising instructions that, when executed by the processor, cause the processor to obtain one or more images and determine one or more compositions of a plurality of regions within the one or more images. Additionally, systems disclosed herein determine a distance between the composition of the region and one or more target vectors for each region in the plurality of regions and select a candidate region from the plurality of regions.

### CROSS-REFERENCE TO RELATED APPLICATIONS

This application claims the benefit of and priority to U.S. Provisional Patent Application No. 63/550,825, filed Feb. 7, 2024, the disclosure of which is hereby incorporated by reference in its entirety for all purposes.

### FIELD OF THE INVENTION

This disclosure relates generally to the methods of identifying and candidate regions of tissue images from a sample based on tissue class compositions for inclusion in a tissue microarray.

### BACKGROUND

Tissue microarrays (TMAs) are invaluable tools for cancer research, enabling the analysis of numerous patient samples simultaneously. TMAs are created from formalin-fixed, paraffin-embedded (FFPE) tumor samples by selecting areas of donor blocks, sampling a cylindrical core out of the donor block, and placing it in a paraffin recipient block. Traditionally, the process of core selection, array design, and TMA construction has been fully manual. Recently, machines have been built to create TMAs from donor blocks (e.g., 3D Histech Grandmaster TMA, others) but the process for selecting cores, assigning donor blocks to recipient arrays, and designing control and layout strategies is still manual, time-consuming, and prone to variability. The need for an automated, intelligent system that can efficiently identify relevant tissue cores and design TMA layouts is evident.

### SUMMARY OF THE INVENTION

Disclosed are methods and systems for selecting one or more candidate regions from a tissue for inclusion in a tissue microarray (TMA). Methods involve determining tissue compositions of regions across the tissue and selecting the candidate regions that have desired tissue compositions for inclusion in a TMA. For example, desired tissue compositions can include one or more tissue classes, examples of which include tumor, stroma, immune, necrosis, other tissue, or combinations thereof. Thus, the disclosed method represents an automated process of selecting candidate regions without involving manual input.

The disclosure generally relates to methods for selecting one or more candidate regions from a tissue for inclusion in a tissue microarray (TMA). Such methods may involve determining tissue compositions of regions of a tissue and selecting the candidate regions that have desired tissue compositions for inclusion in a TMA. The disclosed methods involve an automated process of selecting candidate regions without including manual input. In various embodiments, candidate regions are selected through a similarity assessment that compares the determined tissue composition of a region to one or more target tissue composition vectors. For example, a candidate region can be selected for having a minimum distance between the tissue composition of the region and one or more target composition vectors in comparison to determined distances of other regions.

The advantages of the disclosed methods for selecting candidate regions from a tissue for inclusion in a TMA include the following:
Eliminates the need for manual processing of tissues for generating TMA. The automated process reduces cost of data generation, reduces data variability, and further mitigates batch effects.
Improved and intelligent selection of candidate regions. For example, if the goal is to have a diverse set of regions across a variety of tissue compositions, the disclosed methodology enables accurate identification of regions of varying tissue compositions to be included in one or more TMAs. Alternatively, if the goal is to sample tissue regions of only particular classes (e.g., only tumor class, or only stroma class), the disclosed methodology accurately identifies such undesirable regions are not included on TMAs
Proper selection of controls: the algorithm enables selection of regions of tissue to core that are similar across TMAs, thus providing an experimental control for downstream applications such as imaging.
Improved standardization of core selection across patient tissues, eliminating sampling bias and human error that increase perceived patient samples heterogeneity.
Ability to process large number of patient samples in a high-throughput manner.

Disclosed herein is a method for selecting one or more portions of a tissue, the method comprising: a) obtaining or having obtained one or more images of the tissue; b) determining one or more tissue compositions of a plurality of regions within the one or more images; c) for each region in the plurality of regions, determining a distance between the tissue composition of the region and one or more target composition vectors; and d) selecting a candidate region from the plurality of regions, the candidate region having a minimum distance between the tissue composition of the region and one or more target composition vectors in comparison to determined distances of other regions in the plurality.

In various embodiments, methods further comprise: selecting a portion of the tissue corresponding to the selected candidate region. In various embodiments, methods further comprise: isolating the selected one or more portions of the tissue; and processing the selected one or more portions of the tissue to generate a tissue microarray. In various embodiments, isolating the selected one or more portions comprises sampling one or more tissue cores. In various embodiments, sampling one or more tissue cores comprises sampling one or more cylindrical tissue cores. In various embodiments, processing the selected one or more portions to generate a tissue microarray comprises: generating a paraffin embedding of the one or more selected portions; sectioning the paraffin embedding of the one or more selected portions; and performing tissue staining of the sectioned paraffin embeddings.

In various embodiments, obtaining or having obtained the one or more images comprises: performing staining of one or more slides comprising sections from the tissue; and capturing the one or more images of the one or more slides to obtain the one or more images. In various embodiments, the one or more images comprise one or more hematoxylin and eosin (H&E) stained images. In various embodiments, determining tissue compositions of the plurality of regions within the image comprises deploying a machine learning model. In various embodiments, the machine learning model performs image classification on the one or more images. In various embodiments, the machine learning model performs image classification one or more images using one or more of a convolutional neural network algorithm, logistic regression algorithm, k-nearest neighbor algorithm, support vector machine algorithm, decision tree algorithm, and random forest algorithm. In various embodiments, deploying the machine learning model comprises classifying the one or more images for a plurality of tissue classes. In various embodiments, the plurality of tissue classes comprises at least two, at least three, at least four, or at least five tissue classes. In various embodiments, the plurality of classes comprises two or more of tumor class, stroma class, immune class, necrosis class, or other tissue class. In various embodiments, the plurality of classes comprises each of tumor, stroma, immune, necrosis, or other tissue. In various embodiments, deploying the machine learning model comprises generating tissue composition vectors of one or more regions of an image.

In various embodiments, the tissue composition vector is a vector summarizing the classifications of the plurality of tissue classes for a region. In various embodiments, the tissue composition vector is a 5-element vector summarizing the classifications of tumor, stroma, immune, necrosis, and other tissue for the region. In various embodiments, determining a distance between the tissue composition of the region and the one or more target composition vectors comprises determining the distance between the tissue composition vector of the candidate region and the one or more target composition vectors. In various embodiments, determining the distance comprises determining a cosine distance.

In various embodiments, each of the one or more target composition vectors indicate target percentages of a plurality of tissue classes. In various embodiments, at least one of the target composition vectors indicates a 100% tumor class. In various embodiments, at least one of the target composition vectors is expressed as [1,0,0,0,0], wherein the first entry of the vector corresponds to a tumor tissue class. In various embodiments, at least one of the target composition vectors indicates a 100% stroma class. In various embodiments, at least one of the target composition vectors is expressed as [0,1,0,0,0], wherein the second entry of the vector corresponds to a stroma tissue class. In various embodiments, at least one of the target composition vectors indicates a 50% tumor class and a 50% stroma class. In various embodiments, at least one of the target composition vectors is expressed as [0.5,0.5,0,0,0], wherein the second entry of the vector corresponds to a stroma tissue class. In various embodiments, a first target composition vector indicates a 100% tumor class, a second target composition vector indicates a 100% stroma class, and a third target composition vector indicates a 50% tumor class and a 50% stroma class.

In various embodiments, methods further comprising: generating a ranked list of candidate regions comprising the selected candidate region and one or more additional candidate regions. In various embodiments, generating the ranked list of candidate regions comprises ranking the candidate region and one or more additional candidate regions according to their corresponding determined distances. In various embodiments, the ranked list of candidate regions ranks the candidate region and one or more additional candidate regions in ascending order of cosine distances. In various embodiments, generating the ranked list of candidate regions comprises identifying the one or more additional candidate regions for one or more of a tumor class, stroma class, immune class, necrosis class, other tissue class, tumor-stroma class, or tumor-immune class. In various embodiments, selecting a portion of the tissue corresponding to a candidate region in the ranked list for a tumor tissue class and selecting a portion of the tissue corresponding to a candidate region in the ranked list for a stroma tissue class. In various embodiments, selecting a portion of the tissue corresponding to a candidate region in the ranked list for a tumor-stroma tissue class and selecting a portion of the tissue corresponding to a candidate region in the ranked list for a tumor-immune tissue class. In various embodiments, further comprising selecting five or more candidate regions from the ranked list for a tumor tissue class; selecting five or more candidate regions from the ranked list for a stroma tissue class; selecting five or more candidate regions from the ranked list for a tumor-stroma class; and selecting five or more candidate regions from the ranked list for a tumor-immune class. In various embodiments, the one or more images are captured from the tissue. In various embodiments, the tissue is derived from the subject.

Additionally disclosed herein is a method for a non-transitory computer readable medium comprising instructions that, when executed by a processor, cause the processor to perform any of the disclosed methods. Additionally disclosed herein is a system comprising: a processor; and a non-transitory computer readable medium comprising instructions that, when executed by a processor, cause the processor to perform the method of any one of above methods and embodiments.

### BRIEF DESCRIPTION OF THE DRAWINGS

The foregoing and other objects, features and advantages of the disclosure will become apparent from the following description of preferred embodiments, as illustrated in the accompanying drawings. It is noted that wherever practicable similar or like reference numbers may be used in the figures and may indicate similar or like functionality. For example, a letter after a reference numeral, such as “Tissue Composition 205A,” indicates that the text refers specifically to the element having that particular reference numeral. A reference numeral in the text without a following letter, such as “Tissue Composition 205” refers to any or all of the elements in the figures bearing that reference numeral (e.g. “Tissue Composition 205” in the text refers to reference numerals “Tissue Composition 205A” and/or “Tissue Composition 205B” in the figures).

FIG. 1A shows an example workflow by which a tissue sample is processed for tissue microarray construction, in accordance with an embodiment.

FIG. 1B shows an example workflow involving computational processing of a sample for selection of one or more regions, in accordance with an embodiment.

FIG. 2A is a diagram of an example tissue with multiple identified regions with corresponding tissue compositions, in accordance with an embodiment.

FIG. 2B depicts example target composition vectors, in accordance with an embodiment.

FIG. 3 shows an example workflow for evaluating and selecting region(s), in accordance with an embodiment.

FIG. 4 shows an example workflow of image processing and candidate region selection for tissue microarray construction, in accordance with an embodiment.

FIG. 5 illustrates an example computer for implementing the entities shown in FIGS. 1A-1B, 2A-2B, 3 and 4.

FIG. 6 depicts and outlines example experimental and computational steps involved in processing tissue for TMA construction.

FIG. 7A depicts an example workflow where one or more patient tissue blocks are processed for core selection and TMA construction.

FIG. 7B is an example image that shows the output of the machine learning model on an image of a tissue slice. The circles show cores selected by the disclosed method. The circles are numbered in the order they were selected. Core 0 is the first core chosen, then core 1, and so on. Core 0 has the highest tumor composition of all possible cores, and subsequent cores optimized for tissue composition as well as core placement for optimal packing.

FIG. 7C is the original H&E image of FIG. 7B with annotated cores selected by the core selection algorithm. The annotation text details the type of the core (tumor, tumor-immune, mixed, or random).

FIG. 7D is an example completed TMA.

FIG. 8 shows an example H&E image and comparative annotated tissue compositions either by a pathologist (middle panel) or by the disclosed method (right panel).

### DETAILED DESCRIPTION

### Definitions

Unless otherwise defined herein, scientific and technical terms used in this application shall have the meanings that are commonly understood by those of ordinary skill in the art.

It should be understood that the expression of “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a women or a child).

The phrases “tissue sample” or “tissue” refer to a biological tissue obtained from or derived from a subject. In various embodiments, the tissue can be a three-dimensional tissue structure. Thus, the tissue can be processed through e.g., tissue sectioning and/or tissue staining. In various embodiments, the tissue can be a two-dimensional tissue.

The phrase “tissue composition vector” is described in reference to a region within a tissue and summarizes the tissue classes of the region. In various embodiments, the tissue composition vector specifies compositions for two or more, three or more, four or more, or five or more, six or more, seven or more, eight or more, nine or more, or ten or more tissue classes, examples of which include tumor, stroma, immune, necrosis, and other tissue classes. In various embodiments, the total sum of the values in a tissue composition vector is 100%. Here, the composition of each tissue class in the tissue composition vector is assigned a value less than 100%.

The phrase “target composition vector” refers to a vector specifying desired tissue compositions for one or more tissue classes. In various embodiments, the target composition vector specifies compositions for two or more, three or more, four or more, or five or more tissue classes, examples of which include tumor, stroma, immune, necrosis, and other tissue classes. As an example, a first target composition vector may specify a desired tumor tissue composition whereas a second target composition vector may specify a desired stroma tissue composition. In various embodiments, the total sum of the values in a target composition vector is 100%. Here, the composition of each tissue class in the target composition vector is assigned a value less than 100%.

The phrase “obtaining or having obtained one or more images” encompasses obtaining one or more images by capturing the images using an imaging device. The phrase also encompasses receiving one or more images, e.g., from a third party that captured the images using an imaging device.

The articles “a” and “an” are used herein to refer to one or to more than one (i.e., to at least one) of the grammatical object of the article. By way of example, “an element” means one element or more than one element.

### Overview

Disclosed herein are methods for selecting one or more candidate regions from a tissue for inclusion in a tissue microarray (TMA). Generally, methods involve determining tissue compositions of various regions of a tissue, and selecting the candidate regions that have desired tissue compositions for inclusion in a TMA. In various embodiments, a candidate region is selected based on a determined distance between the tissue composition of the region and one or more target composition vectors representing desired tissue compositions. In various embodiments, different candidate regions can be selected that have different tissue compositions and therefore, the resulting TMA can have a set of candidate regions representing a diverse set of tissue compositions. Thus, subsequent downstream analysis of regions included in the TMA can provide readouts for the set of candidate regions with diverse tissue compositions.

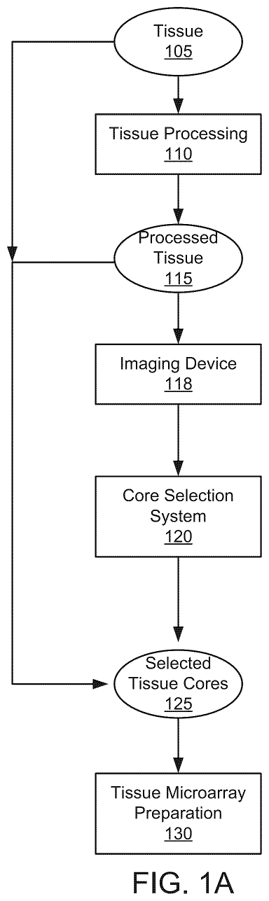

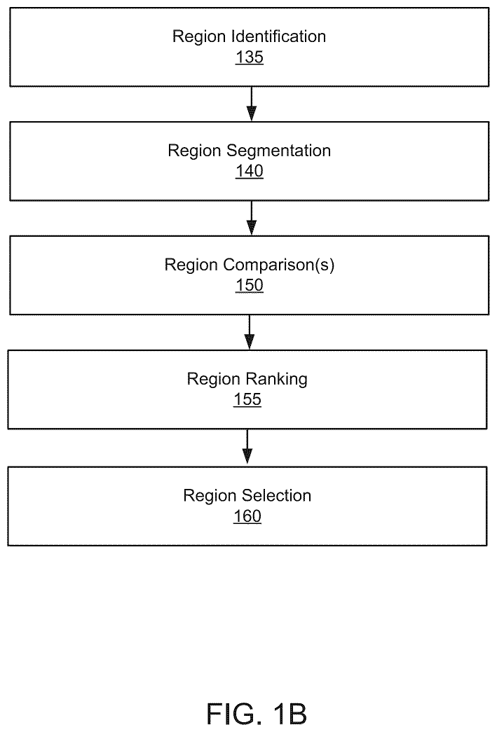

Reference is now made to FIG. 1A, which shows an example flow diagram for selecting tissue cores for TMA preparation from a tissue sample. As shown in FIG. 1A, step 105 involves obtaining a tissue. In various embodiments, the tissue can be obtained from a subject, such as a mammalian subject (e.g., a mouse, rat, pig, monkey, or human) subject. In various embodiments, the subject is a cancer subject (e.g., a subject previously diagnosed with cancer). Thus, the tissue can be a cancer biopsy obtained from the cancer subject. In various embodiments, the subject is suspected of having cancer but has not previously been diagnosed with cancer. The tissue can be a biopsy obtained from the subject suspected of having cancer. In various embodiments, the subject is a healthy subject. Thus, the tissue can be a healthy tissue obtained from the healthy subject.

In various embodiments, the obtained tissue can be of a tissue type from one or more organ systems including but not limited to: muscle, reproductive, integumentary, skeletal, kidney, immune, parathyroid, respiratory, digestive, lymphatic, circulatory, urinary, nervous, endocrine, cardiovascular, and/or excretory. In various embodiments, the tissue can include any one of hair, skin, nails, cartilage, bones, joints, skeletal muscle, tendons, brain, spinal cord, peripheral nerves, pituitary gland, thyroid gland, pancreas, adrenal gland, testes, ovaries, heart, blood vessels, thymus, lymph nodes, spleen, lymphatic vessels, nasal passage, trachea, lung, stomach, liver, gall bladder, large intestine, small intestine, kidneys, urinary bladder, epididymis, mammary glands, and/or uterus. The aforementioned tissue can be acquired through multiple methods, including but not limited to, biopsy (including but not limited to: bone marrow aspiration, cardiac, core, endometrial, endoscopic, excisional and incisional, fine-needle aspiration, lymph node, needle, open, punch, sentinel lymph node, shave, and skin), autopsy, and necropsy from a subject. In addition, the tissue sample may also feature pathological cellular features associated with a range of conditions, including but not limited to cancer, inflammation, and/or necrosis.

As shown in FIG. 1A, step 110 involves tissue processing. In various embodiments, tissue processing involves one or more of tissue fixing, tissue sectioning, and/or tissue staining. In various embodiments, tissue processing involves each of tissue fixing, tissue sectioning, and/or tissue staining. In various embodiments, tissue processing can involve performing, more than once, any or all of the steps of tissue fixing, tissue sectioning, and/or tissue staining. For example, a tissue 105 obtained from a subject can undergo tissue processing at step 110 including tissue fixing, tissue sectioning, and/or tissue staining. Subsequently, the tissue can be unfixed (e.g., by melting away the fixative), re-embedded, and further sectioned and/or stained for other investigatory purposes.

In various embodiments, processing the tissue at step 115 involves preparing a tissue specimen that can be readily imaged e.g., by the imaging device 118. For example, processing the tissue at step 115 can involve sectioning the tissue to obtain tissue sections that can be mounted onto microscope slides. The tissue sections can be further stained such that characteristics of interest can be visually apparent when imaged e.g., by the imaging device 118. Example stains include antibody stains, fluorescent tags, or hematoxylin and eosin (H&E) stains. Further details of processing tissues are described herein.

The imaging device 118 captures one or more images of the processed tissue 115. In various embodiments, the imaging device 118 encompasses a microscope and a camera system that allows for the digital capture of optical data in the form of images. In various embodiments, the one or more images can be captured using one or more of the following imaging modalities including fluorescent microscopy, confocal microscopy, super-high-resolution microscopy, in vivo two photon microscopy, electron microscopy (e.g., scanning electron microscopy or transmission electron microscopy), atomic force microscopy, bright field microscopy, optical microscopy, or phase contrast microscopy. In particular embodiments, the imaging device 118 is an optical microscope. In particular embodiments, the imaging device 118 is a bright field microscope. In particular embodiments, the imaging device 118 is a phase contrast field microscope.

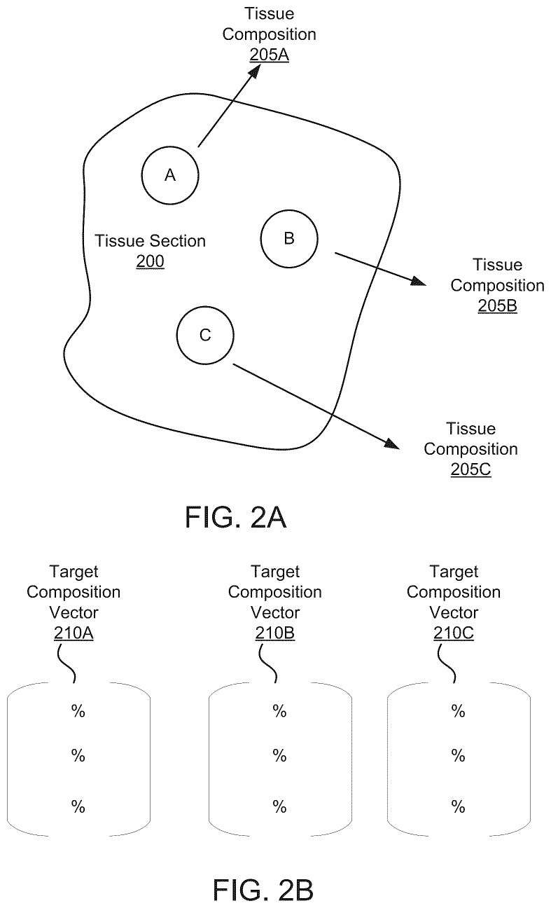

Attention is now directed towards the core selection system 120. Generally, the core selection system 120 analyzes the image of the processed tissue 115 captured by the imaging device 118 and selects candidate regions for inclusion in a TMA. The core selection system 120 performs a computational analysis of the image that involves one or more of identifying regions in the tissue, segmenting regions within the tissue, comparing compositions of the regions, ranking candidate regions, and selecting candidate regions for inclusion in a TMA. In various embodiments, the core selection system 120 further performs a region identification step that identifies a set of regions in the tissue. Generally, the core selection system 120 determines tissue compositions for each of one or more regions in the tissue and compares each of the tissue compositions to one or more target composition vectors representing desired tissue compositions. The core selection system 120 can select different candidate regions with tissue compositions that are closest to the desired tissue compositions. For example, the core selection system 120 determines distances between tissue compositions of regions of the tissue and one or more target composition vectors and selects candidate regions with the lowest determined distances. The candidate regions are therefore selected for inclusion in the TMA. Further details of the core selection system 120 are described herein with respect to FIGS. 2A-2B, 3, and 4.

As shown in FIG. 1A, the selected tissue cores 125, which include the candidate regions identified by the core selection system 120, are used for TMA preparation at step 130. TMA preparation refers to a technique wherein samples from different sources (“donors”) are transferred to a single “recipient” block. This TMA block then allows for the efficient processing of biomarkers (protein, RNA, etc.) within numerous donor tissue samples.

In various embodiments, step 130 can involve isolating the selected tissue cores 125 from the processed tissue 115 or from the originally obtained tissue 105. In various embodiments, isolating the selected tissue cores 125 involves sampling one or more tissue cores corresponding to the selected tissue cores 125. For example, sampling one or more tissue cores can refer to the process by which candidate region is isolated through a set geometric shape (e.g., using a hollow needle having the geometric shape). In various embodiments, the geometric shape can include, but not limited to, a circle, square, rectangle, triangle, or polygon. In particular embodiments, the geometric shape of a tissue core is a circle (thereby generating a 3D cylindrical tissue core).

In various embodiments, a tissue core has a diameter between 0.2 mm to 5 mm. In various embodiments, the tissue core has a diameter between 0.3 and 4.5 mm, between 0.4 and 4.0 mm, between 0.5 and 3.5 mm, between 0.6 and 3.0 mm, between 0.7 and 2.5 mm, between 0.8 and 2.0 mm, or between 0.9 and 1.5 mm. In various embodiments, the tissue core has a diameter of 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, or 5.0 mm. In particular embodiments, the tissue core has a diameter of 0.6 mm.

In various embodiments, the TMA block may include between 100 and 5000 cores. In various embodiments, the TMA block may include between 150 and 4900 cores, between 200 and 4800 cores, between 250 and 4700 cores, between 300 and 4600 cores, between 350 and 4500 cores, between 400 and 4400 cores, between 450 and 4300 cores, between 500 and 4200 cores, between 550 and 4100 cores, between 600 and 4000 cores, between 650 and 3900 cores, between 700 and 3800 cores, between 750 and 3700 cores, between 800 and 3600 cores, between 850 and 3500 cores, between 900 and 3400 cores, between 1000 and 3300 cores, between 1050 and 3200 cores, between 1100 and 3100 cores, between 1150 and 3000 cores, between 1200 and 2900 cores, between 1250 and 2800 cores, between 1300 and 2700 cores, between 1350 and 2600 cores, between 1400 and 2500 cores, between 1450 and 2400 cores, between 1500 and 2300 cores, between 1550 and 2200 cores, between 1600 and 2100 cores, between 1650 and 2000 cores, or between 1700 and 1900 cores. In various embodiments, the TMA block may include 100 cores, 150 cores, 200 cores, 250 cores, 300 cores, 350 cores, 400 cores, 450 cores, 500 cores, 550 cores, 600 cores, 650 cores, 700 cores, 750 cores, 800 cores, 850 cores, 900 cores, 950 cores, 1000 cores, 1100 cores, 1200 cores, 1300 cores, 1400 cores, 1500 cores, 1600 cores, 1700 cores, 1800 cores, 1900 cores, 2000 cores, 2500 cores, 3000 cores, 3500 cores, 4000 cores, 4500 cores, or 5000 cores.

In various embodiments, step 130 involves melting and sectioning the selected tissue cores 125 onto the TMA block. In various embodiments, step 130 involves generating a paraffin embedding of the one or more selected tissue cores 125, sectioning the paraffin embedding of the one or more selected tissue cores 125; and performing tissue staining of the sectioned paraffin embeddings. In various embodiments, step 130 is performed via an automated process. For example, step 130 can be performed using a dedicated tissue microarrayer that automatically isolates the selected tissue cores 125 from the processed tissue 115 or tissue 105 onto the TMA block. Example tissue microarrayers include the TMA Grand Master (3DHISTECH), TMA Master II (3DHISTECH), TMA Master (3DHisTech), AutoTiss 10C (SciPro), and Galileo (TMAatic).

### Methods for Selecting Candidate Regions in a Tissue

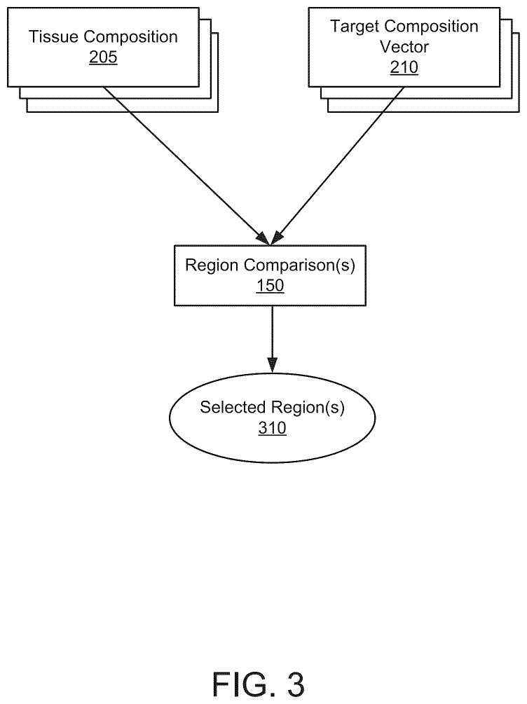

Attention is now directed towards FIG. 1B, which shows an example workflow involving computational processing of a sample for selection of one or more regions, in accordance with an embodiment. Reference will be additionally made to FIGS. 2A, 2B, and 3. FIG. 2A is a diagram of an example tissue with multiple identified regions with corresponding tissue compositions, in accordance with an embodiment. FIG. 2B depicts example target composition vectors, in accordance with an embodiment. FIG. 3 shows an example workflow for evaluating and selecting region(s), in accordance with an embodiment.

Referring to FIG. 1B, steps 135-160 describe one embodiment in which the core selection system 120 (shown in FIG. 1A) selects candidate regions for inclusion in a TMA. In various embodiments, one or more of the steps shown in FIG. 1B can be excluded. For example, in various embodiments, step 135 involving region identification and/or step 155 involving region ranking need not be performed. In other words, step 135 and step 155 can be optional steps for selecting candidate regions for inclusion in a TMA.

Step 135 involves performing region identification to identify, within an image, one or more regions of a tissue. In various embodiments, step 135 involves identifying 2 or more regions, 3 or more regions, 4 or more regions, 5 or more regions, 6 or more regions, 7 or more regions, 8 or more regions, 9 or more regions, 10 or more regions, 11 or more regions, 12 or more regions, 13 or more regions, 14 or more regions, 15 or more regions, 16 or more regions, 17 or more regions, 18 or more regions, 19 or more regions, 20 or more regions, 21 or more regions, 22 or more regions, 23 or more regions, 24 or more regions, 25 or more regions, 26 or more regions, 27 or more regions, 28 or more regions, 29 or more regions, 30 or more regions, 31 or more regions, 32 or more regions, 33 or more regions, 34 or more regions, 35 or more regions, 36 or more regions, 37 or more regions, 38 or more regions, 39 or more regions, 40 or more regions, 41 or more regions, 42 or more regions, 43 or more regions, 44 or more regions, 45 or more regions, 46 or more regions, 47 or more regions, 48 or more regions, 49 or more regions, 50 or more regions, 51 or more regions, 52 or more regions, 53 or more regions, 54 or more regions, 55 or more regions, 56 or more regions, 57 or more regions, 58 or more regions, 59 or more regions, 60 or more regions, 61 or more regions, 62 or more regions, 63 or more regions, 64 or more regions, 65 or more regions, 66 or more regions, 67 or more regions, 68 or more regions, 69 or more regions, 70 or more regions, 71 or more regions, 72 or more regions, 73 or more regions, 74 or more regions, 75 or more regions, 76 or more regions, 77 or more regions, 78 or more regions, 79 or more regions, 80 or more regions, 81 or more regions, 82 or more regions, 83 or more regions, 84 or more regions, 85 or more regions, 86 or more regions, 87 or more regions, 88 or more regions, 89 or more regions, 90 or more regions, 91 or more regions, 92 or more regions, 93 or more regions, 94 or more regions, 95 or more regions, 96 or more regions, 97 or more regions, 98 or more regions, 99 or more regions, or 100 or more regions.

In various embodiments, step 135 can involve randomly identifying regions within the tissue such that that identified regions are non-overlapping. In various embodiments, step 135 involves identifying regions to maximize the total number of non-overlapping regions within the tissue. As one example, maximizing the total number of non-overlapping regions can ensure that all portions of the tissue are adequately covered by at least one region. As another example, if a large number of TMAs are to be generated, then a maximum number of non-overlapping regions within the tissue can be identified to ensure that all TMAs can be saturated. In various embodiments, non-overlapping regions within the tissue are separated by at least 0.1 mm to ensure that adjacent regions, if selected, can each be individually isolated without impacting the structural integrity of the adjacent region. In various embodiments, non-overlapping regions within the tissue are separated by at least 0.2 mm, at least 0.3 mm, at least 0.4 mm, at least 0.5 mm, at least 0.6 mm, at least 0.7 mm, at least 0.8 mm, at least 0.9 mm, or at least 1 mm to ensure that adjacent regions, if selected, can each be individually isolated without impacting the structural integrity of the adjacent region. In various embodiments, non-overlapping regions within the tissue are separated by at least 0.4 mm to ensure that adjacent regions, if selected, can each be individually isolated without impacting the structural integrity of the adjacent region.

Referring to FIG. 2A, it shows an example image of a tissue section 200. In particular embodiments, the image is a H&E stained image of a tissue section 200, but other types of images can also be used, as described herein. Tissue section 200 shows three identified regions, labeled as “A”, “B”, and “C”. In various embodiments, fewer or more regions in a tissue section can be identified. Furthermore, each region “A”, “B”, and “C” are shown as circular regions. In some embodiments, regions can have a different geometric shape, such as any of a square, rectangle, triangle, or polygon.

Returning to step 140 in FIG. 1B, step 140 involves performing region segmentation to determine distinct tissue classes within the one or more regions of the tissue. Example tissue classes includes, tumor, stroma, immune, necrosis, and other, however this approach can be expanded to other tissue classes. In various embodiments, example tissue classes can be relevant to liver tissue. For example, liver tissue classes can include different liver cells, such as one or more of hepatocytes, Kupffer cells, Ito cells (hepatic stellate cells), and liver endothelial cells (sinusoidal endothelial cells). Each cell-type carries out unique functions and can be differentially identified through imaging and segmentation/classification approaches. As another example, liver tissue classes can involve structures of the liver including one or more of sinusoids, bile canaliculi, bile ducts, portal triad, central vein, periportal zone, perivenous zone, and Space of Disse.

In various embodiments, performing region segmentation involves implementing a trained machine learning model. In such embodiments, the machine learning model recognizes distinct visual properties in regions of the tissue and determines proportions of each tissue class in the region. For example, given N different tissue classes (e.g., two or more, three or more, four or more, or five or more, six or more, seven or more, eight or more, nine or more, or ten or more tissue classes), the machine learning model can analyze an image and outputs a prediction for each of the N different tissue classes. Further details of example machine learning models are described herein.

Referring to FIG. 2A, region segmentation is performed on each of the regions “A”, “B”, and “C” to determine tissue composition 205A, tissue composition 205B, and tissue composition 205C, respectively. In various embodiments, a trained machine learning model analyzes region “A” (e.g., the machine learning model receives as input the image pixels of region “A”) and predicts the tissue composition 205A. Additionally, the trained machine learning model analyzes region “B” (e.g., the machine learning model receives as input the image pixels of region “B”) and predicts the tissue composition 205B, and the trained machine learning model analyzes region “C” (e.g., the machine learning model receives as input the image pixels of region “C”) and predicts the tissue composition 205C. Here, each tissue composition 205 may indicate a percentage for each of N different tissue classes. As one example, each tissue composition 205 may indicate a percentage for one or more of a stroma, tumor, immune, necrosis, and other tissue class.

Returning to FIG. 1B, upon completion of region segmentation at step 140, step 150 involves performing region comparison(s) to identify regions with tissue compositions that are most similar with a desired target composition vector. Here, the target composition vector can be a pre-set vector indicating desired tissue class proportions. For example, if a tissue microarray (or a portion of a tissue microarray) is to be used for analyzing tumor tissue, then the target composition vector can be set to include a high tumor proportion (e.g., greater than 50%, greater than 60%, greater than 70%, greater than 80%, greater than 90%, greater than 95%, or greater than 99% tumor proportion). As another example, if a tissue microarray (or a portion of a tissue microarray) is to be used for analyzing stromal tissue, then the target composition vector can be set to include a high stromal proportion (e.g., greater than 50%, greater than 60%, greater than 70%, greater than 80%, greater than 90%, greater than 95%, or greater than 99% stromal proportion). As another example, if a tissue microarray (or a portion of a tissue microarray) is to be used for analyzing necrotic tissue, then the target composition vector can be set to include a high necrosis proportion (e.g., greater than 50%, greater than 60%, greater than 70%, greater than 80%, greater than 90%, greater than 95%, or greater than 99% necrosis proportion). As another example, if a tissue microarray (or a portion of a tissue microarray) is to be used for analyzing immune tissue, then the target composition vector can be set to include a high immune proportion (e.g., greater than 50%, greater than 60%, greater than 70%, greater than 80%, greater than 90%, greater than 95%, or greater than 99% immune proportion).

In various embodiments, target composition vectors can be preset to indicate combinations of desired tissue classes. In various embodiments, target composition vectors can be preset to include a mixture of proportions for two or more of tumor, stroma, immune, necrosis, or other tissue. In various embodiments, target composition vectors can be preset to include a mixture of proportions for three or more of, four or more of, or each of tumor, stroma, immune, necrosis, or other tissue. For example, a tissue composition vector can indicate a 50% proportion of a first tissue class and a 50% proportion of a second tissue class. As another example, a tissue composition vector can indicate a 50% proportion of a first tissue class, a 25% proportion of a second tissue class, and a 25% proportion of a third tissue class. Additional proportions of different tissue classes can be implemented. In various embodiments, the different tissue composition vectors can be tuned and/or modified to ensure that selected candidate regions for the tissue microarray can span various types of tissue compositions. Thus, this ensures that a single tissue microarray can provide readouts across the full range of tissue compositions.

Reference is now made to FIG. 2B, which depicts example target composition vectors, in accordance with an embodiment. Specifically, FIG. 2B shows three target composition vectors (e.g., target compositions vectors 210A, 210B, and 210C), but in various embodiments, fewer or additional target composition vectors can be implemented. For example, in various embodiments, there may be four, five, six, seven, eight, nine, ten, eleven, twelve, thirteen, fourteen, fifteen, sixteen, seventeen, eighteen, nineteen, or twenty target composition vectors.

Furthermore, FIG. 2B shows that a target composition vector 210 includes three entries (as denoted by the three “%” symbols). Generally, the number of entries in a target composition vector 210 will align with the number of entries in the tissue composition 205. In particular embodiments, each tissue composition 205 and each target composition vector 210 will include three entries. In particular embodiments, each tissue composition 205 and each target composition vector 210 will include four entries. In particular embodiments, each tissue composition 205 and each target composition vector 210 will include five entries.

In various embodiments, the target composition vector indicates 100% tumor. For example, the target composition vector can be denoted as [1, 0, 0, 0, 0], where the first entry in the vector refers to a tumor class, and the other four entries refer to other tissue classes. In various embodiments, the target composition vector indicates 100% stroma. For example, the target composition vector can be denoted as [0, 1, 0, 0, 0], where the second entry in the vector refers to a stroma class, and the other four entries refer to other tissue classes. In various embodiments, the target composition vector indicates 100% immune. For example, the target composition vector can be denoted as [0, 0, 1, 0, 0], where the third entry in the vector refers to an immune class, and the other four entries refer to other tissue classes. In various embodiments, the target composition vector indicates 100% necrosis. For example, the target composition vector can be denoted as [0, 0, 0, 1, 0], where the fourth entry in the vector refers to a necrosis class, and the other four entries refer to other tissue classes. In various embodiments, the target composition vector indicates 100% other class. For example, the target composition vector can be denoted as [0, 0, 0, 0, 1], where the fifth entry in the vector refers to the “other” class, and the other four entries refer to tissue classes such as tumor, stroma, immune, and necrosis.

In various embodiments, other combinations and other proportions of various tissue classes in a target composition vector are also possible. As one example, the target composition vector indicates 50% tumor and 50% stroma. The target composition vector can be denoted as [0.5, 0.5, 0, 0, 0], where the first entry refers to a tumor class, the second entry refers to a stroma class, and the other three entries refer to other tissue classes. As another example, the target composition vector indicates 50% tumor and 50% immune. The target composition vector can be denoted as [0.5, 0, 0.5, 0, 0], where the first entry refers to a tumor class, the third entry refers to a immune class, and the other three entries refer to other tissue classes. As another example, the target composition vector indicates 50% tumor and 50% necrosis. The target composition vector can be denoted as [0.5, 0, 0, 0.5, 0], where the first entry refers to a tumor class, the fourth entry refers to a stroma class, and the other three entries refer to other tissue classes.

After region segmentation 140, step 150 involves performing region comparison(s). For example, as shown in FIG. 3, multiple tissue compositions 205 can be compared to multiple tissue composition vectors 210 at step 150. In various embodiments, a subset of the tissue compositions 205 are compared to a subset of the tissue composition vectors 210. In particular embodiments, every tissue composition 205 is compared to every tissue composition vector 210.

In various embodiments, comparing a tissue composition to a tissue composition vector comprises determining a distance between the tissue composition and the target composition vector. The distance represents a measure of similarity between the tissue composition and the target composition vector. In various embodiments, the distance can be a measure of dissimilarity between vectors. Example distances include a cosine distance, a Euclidean distance, a Manhattan distance, a Sorensen-dice coefficient (also referred to as a dice coefficient), or a Jaccard similarity. In various embodiments, a smaller distance represents a higher similarity between the tissue composition and the target composition vector. In various embodiments, a larger distance represents a reduced similarity between the tissue composition and the target composition vector. For example, assume a first distance between a first tissue composition and a target composition vector that is smaller than a second distance between a second tissue composition and the target composition vector. In this scenario, the first tissue composition would be more similar to the target composition vector in comparison to the second tissue composition.

In various embodiments, the distance between the tissue composition and the target composition vector is a cosine distance. The cosine distance is a similarity measure based on the cosine of the angle between the two vectors. Here, the cosine distance between the tissue composition (A) and target composition vector (B) can be denoted as:

$$C osine distance = \frac{A \cdot B}{‖ A ‖ ‖ B ‖}$$

where A·B represents the dot product between the tissue composition (A) and target composition vector (B), and where ∥A∥|B∥ represents the product of the magnitudes of the tissue composition (A) and target composition vector (B).

In various embodiments, after performing region comparison, step 155 involves performing region ranking. Regions can be ranked either in decreasing or increasing similarity. Generally, regions of a tissue are ranked according to their determined distances. For example, regions of tissue with a smaller distance are ranked more highly in comparison to regions of tissue with a larger distance. Regions of tissue with a smaller distance that are highly ranked can therefore be selected as candidate regions for inclusion in a TMA.

In various embodiments, a ranking is generated for each target composition vector. For example, assume X different target composition vectors. In this scenario, X different rankings are generated, where each rank list includes tissue compositions that are each ranked according to the determined distances between the tissue composition of each region and the target composition vector.

As a specific example, a first target composition vector may be a 100% tumor target composition vector denoted as [1,0,0,0,0]. A second target composition vector may be a 100% stroma target composition vector denoted as [0,1,0,0,0]. A third target composition vector may be a 50% tumor, 50% stroma target composition vector denoted as [0.5,0.5,0,0,0]. A fourth target composition vector may be a 50% tumor, 50% immune target composition vector denoted as [0.5,0,0.5,0,0]. In this scenario, step 155 involves generating four different rank lists in which the regions most highly ranked on each rank list have tissue compositions that have the lowest distance to the corresponding target composition vector.

Returning to FIG. 1B, step 160 involves performing region selection. Here, the selected regions are candidate regions for inclusion in a TMA. In various embodiments, the selected candidate region has a minimum distance between the tissue composition of the region and one or more target composition vectors in comparison to determined distances of other regions. For example, given a ranking of regions for a target composition vector, the selected candidate region may be the highest ranked (e.g., region with a minimum distance).

In various embodiments, step 160 involves selecting the regions that are most highly ranked. For example, step 160 can involve selecting the region that is most highly ranked for a particular tissue composition vector. In various embodiments, step 160 can involve selecting each region that is most highly ranked for each tissue composition vector. For example, given a 100% tumor target composition vector and a 100% stroma target composition vector, step 160 can involve selecting a first region that is highest ranked for the 100% tumor target composition vector as a candidate region and further selecting a second region that is highest ranked for the 100% stroma target composition vector as another candidate region. Furthermore, given a scenario where there is an additional 50% tumor 50% stroma target composition vector and/or a 50% tumor 50% immune target composition vector, step 160 can involve selecting an additional region that is highest ranked for the 50% tumor 50% stroma target composition vector as a candidate region and/or selecting an additional region that is highest ranked for the 50% tumor 50% immune target composition vector as another candidate region.

In various embodiments, step 160 can be repeated to select additional regions as candidate regions. For example, given that a first region with the minimum distance has been selected as a candidate region, step 160 can be repeated to selected a second region with the next minimum distance. Given a ranking of regions for a target composition vector, the second region may be the next highest ranked region (e.g., region with the second lowest minimum distance).

In various embodiments, step 160 can be further repeated until a threshold condition is met. In some embodiments, the threshold condition is a threshold number of regions from a ranked list. In various embodiments, the threshold number of regions from a ranked list is two or more regions, three or more regions, four or more regions, five or more regions, six or more regions, seven or more regions, eight or more regions, nine or more regions, or ten or more regions. In particular embodiments, the threshold number of regions from a ranked list is three regions. In particular embodiments, the threshold number of regions from a ranked list is six regions. For example, given any of a 100% tumor target composition vector, a 100% stroma target composition vector, a 50% tumor 50% stroma target composition vector, or a 50% tumor 50% immune target composition vector, the threshold number of regions may be two or more regions, three or more regions, four or more regions, five or more regions, six or more regions, seven or more regions, eight or more regions, nine or more regions, or ten or more regions. In particular embodiments, given any of a 100% tumor target composition vector, a 100% stroma target composition vector, a 50% tumor 50% stroma target composition vector, or a 50% tumor 50% immune target composition vector, the threshold number of regions is five or more regions. For example, the top 5 ranked regions for each target composition vector can be selected as a candidate region. As another example, the top 6 ranked regions for each target composition vector can be selected as a candidate region.

### Example Flow Diagram for Selecting Candidate Regions for Tissue Microarray

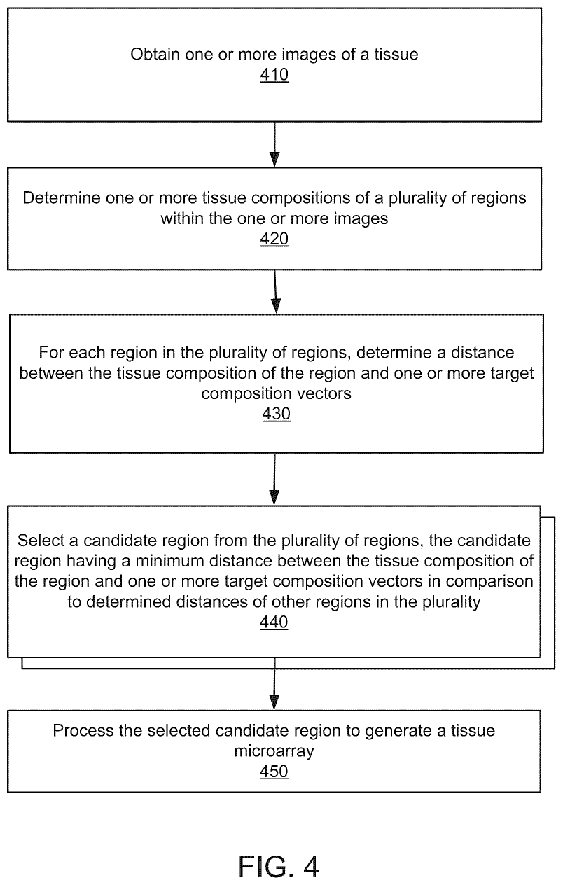

Attention is now drawn to FIG. 4, which shows an example workflow of image processing and candidate region selection for tissue microarray construction, in accordance with an embodiment.

Step 410 involves obtaining or having obtained one or more images of a tissue.

Step 420 involves determining one or more tissue compositions of a plurality of regions within the one or more images. For example, step 420 may involve determining a tissue composition vector for each region that summarizes the tissue classes of the region.

Step 430 involves determining, for each region in the plurality of regions, a distance between the tissue composition of the region and one or more target composition vectors. In particular embodiments, the distance is a cosine distance between the tissue composition vector of the region and a target composition vector.

Step 440 involves selecting a candidate region from the plurality of regions, the candidate region having a minimum distance between the tissue composition of the region and one or more target composition vectors in comparison to determined distances of other regions in the plurality. In various embodiments, step 440 can be repeated to identify additional candidate regions. For example, having identified a first candidate region, step 440 is repeated to identify an additional candidate region that has the next minimum distance between the tissue composition of the region and one or more target composition vectors. As another example, having identified a first candidate region, step 440 is repeated to identify an additional candidate region that has a minimum distance between the tissue composition of the region and a different target composition vector.

Step 450 involves processing the selected region(s) to generate a tissue microarray.

### Example Machine Learning Models

As disclosed herein, methods for selecting one or more candidate regions from a tissue for inclusion in a tissue microarray (TMA) may involve implementing a machine learning model for performing region segmentation e.g., for determining tissue compositions of regions of a tissue. Generally, the machine learning model is trained using training data to predict compositions of various tissue classes for individual regions in a tissue. Given an image of a tissue, the machine learning model can generate a prediction of the various tissue classes for the region. For example, given an image of a tissue, the machine learning model generates a prediction vector reflecting the tissue composition of the region. Such a prediction vector can include percentages for one or more tissue classes e.g., tumor, stroma, necrosis, non-tissue, and other tissue classes.

In various embodiments, a machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks).

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, transfer, multi-task learning, or any combination thereof.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In various embodiments, the model parameters of the machine learning model can be adjusted to minimize an error representing the difference between a prediction of the machine learning model and a reference ground truth of the training data. For example, the model parameters of the machine learning model can be adjusted to minimize a loss function, where the loss value represents a penalty that is the difference between the prediction of the machine learning model and the reference ground truth. In various embodiments, the reference ground truth data indicates the known tissue composition of a region of tissue. Here, the ground truth data can be a composition vector that identifies the known percent composition of one or more tissue classes for the region of the tissue. For example, given N different tissue classes, the ground truth can be expressed as a composition vector denoted as: [a %, b %, c % . . . n %] where each of a, b, c . . . n represent numerical percentages.

In various embodiments, ground truth data can be labeled by an expert (e.g., an expert pathologist) who analyzes the region of the tissue. Therefore, by training the machine learning model to minimize the error, the machine learning model can more accurately predict tissue compositions of regions in tissues that have been previously unencountered (e.g., for purposes of selecting candidate regions for inclusion in a TMA, as is disclosed herein).

### Example Tissues and Methods for Processing Tissues

As disclosed herein, tissues are obtained and processed e.g., for selection of candidate regions to be included in a tissue microarray (TMA). As described in reference to FIG. 1A, a tissue 105 is obtained and processed at step 110 to produce processed tissue 115. This section describes steps in the tissue processing 110, including one or more of tissue sectioning, tissue fixing, and/or tissue staining.

In various embodiments, tissues are fixed and sectioned. In various embodiments, the tissue can be fixed using various fixation methods including, but not limited to, formalin, formaldehyde, and/or ethanol fixation. Sectioning can be achieved through multiple modalities (cryostat, microtome, or vibratome) and generally involves the slicing of tissue at various thickness that can optionally be embedded in a medium such as paraffin, optimal cutting temperature, agarose, celloidin, and other embedding media. In particular embodiments, tissues are fixed, paraffin embedded, and cut. Tissues may be fixed by perfusing the tissue using a formaldehyde fixation solution. Tissues can be dehydrated by immersing them consecutively in increasing concentrations of ethanol (e.g., 70%, 90%, 100% ethanol) and then immersed in xylene. Tissues can be embedded in paraffin and then cut into tissue sections (e.g., 5-15 microns in thickness). This can be accomplished using a microtome. Tissue sections are mounted onto histological slides, and then dried.

In various embodiments, tissues undergo a staining process. The phrase “tissue staining” refers to the process by which a tissue section is provided a staining agent that enable visualizations certain tissue characteristics. In various embodiments, tissue staining occurs after tissue fixation and/or tissue sectioning. Thus, in such embodiments, a mounted tissue section on a histological slide undergoes tissue staining. In various embodiments, tissue staining can involve a chemical and/or physical modification to the tissue. Example tissue staining processes can include, but are not limited to, methods such immunohistochemistry staining, H&E staining (where hematoxylin stains the acidic components of the cell and eosin the basic component of the cell, wherein acidic and basic refer to pH conditions), and immunofluorescence staining.

For immunohistochemistry staining, methods may involve providing primary/secondary antibodies for enabling visualization of certain tissue characteristics of interest (e.g., cells, proteins, and/or biomarkers of interest). The primary/secondary antibodies may exhibit binding specificity for the targets of interest. In various embodiments, tissues can be treated using blocking buffer to block for non-specific staining between a primary antibody and the tissue. Example blocking buffer can include 1% horse serum in phosphate buffered saline. Primary antibodies are diluted to appropriate dilutions and applied to the tissue sections. Tissue slices are washed, then incubated with a secondary antibody specific for the primary antibody. Tissue slices are washed, and then mounted for imaging. Additional methods for performing immunohistochemistry are described in further detail in Simon et al, BioTechniques, 36(1):98 (2004) and Haedicke et al., BioTechniques, 35(1):164 (2003), each of which is hereby incorporated by reference in its entirety. In various embodiments, immunohistochemistry can be automated using commercially available instruments, such as the Benchmark ULTRA system available from the Roche Group.

For H&E staining, instead of primary/secondary antibodies, hematoxylin solution can be applied to tissue sections (e.g., tissue sections mounted on slides), rinsed in distilled water and placed in alcohol (e.g., 95% alcohol). Counterstaining occurs with Eosin solution exposure to the tissue section and is subsequently dehydrated through one or more alcohol rinses (e.g., 95 and 100% alcohol rinses) and one or more xylene rinses.

For immunofluorescence staining, tissue sections can be stained for particular targets (e.g., proteins, biomarkers) of interest. Methods may involve providing fluorescently labeled primary/secondary antibodies exhibiting binding specificity for the targets of interest. In various embodiments, tissues are treated using blocking buffer to block for non-specific staining between a primary antibody and the tissue. Example blocking buffer can include 1% horse serum in phosphate buffered saline. Primary antibodies conjugated to fluorescent proteins are diluted to appropriate dilutions and applied to the tissue sections. Tissue slices are washed, and then mounted for imaging.

### Example Cancers

In various embodiments, methods disclosed herein are useful for selecting one or more candidate regions from a tumor tissue for inclusion in a tissue microarray (TMA). The tumor tissue may be obtained from a subject with a cancer. In various embodiments, the cancer is any of an acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

### System Embodiments

The methods of the disclosure, including the methods for selecting one or more candidate regions from a tissue for inclusion in a tissue microarray (TMA) are, in some embodiments, performed on one or more computers. For example, the steps shown in FIG. 1B that may be performed by the core selection system 120 shown in FIG. 1A may be performed on one or more computers.

In various embodiments, methods for selecting one or more candidate regions from a tissue for inclusion in a tissue microarray (TMA) can be implemented in hardware or software, or a combination of both. In some embodiments, a machine-readable storage medium is provided, the medium comprising a data storage material encoded with machine readable data which, when using a machine programmed with instructions for using said data, is capable of displaying data (e.g., images of tissues and/or candidate regions) and results. Such data can be used for a variety of purposes, such as for isolating the tissue cores and preparing a TMA. Methods of the disclosure can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, methods for selecting one or more candidate regions from a tissue for inclusion in a tissue microarray (TMA) are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

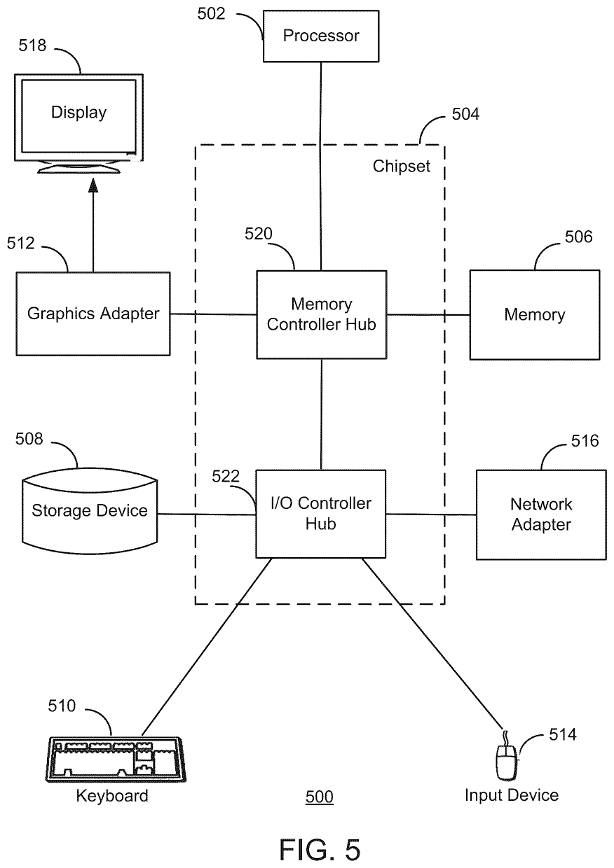

FIG. 5 illustrates an example computer for implementing the entities shown in FIGS. 1A-1B, 2A-2B, 3 and 4. In particular embodiments, the example computer 500 can represent the core selection system shown in FIG. 1A. The computer 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, an input device 514, and network adapter 516 are coupled to the I/O controller hub 522. Other embodiments of the computer 500 may have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input interface 514 is a touch-screen interface, a mouse, track ball, or other type of pointing device, a keyboard, or some combination thereof, and is used to input data into the computer 500. In some embodiments, the computer 500 may be configured to receive input (e.g., commands) from the input interface 514 via gestures from the user. The graphics adapter 512 displays images and other information on the display 518. The network adapter 516 couples the computer 500 to one or more computer networks.

The computer 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 500 used by the entities of the core selection system 120 shown in FIG. 1A, can vary depending upon the embodiment and the processing power required by the entity. For example, the core selection system 120 shown in FIG. 1A, can run in a single computer 500 or multiple computers 500 communicating with each other through a network such as in a server farm. The computers 500 can lack some of the components described above, such as graphics adapters 512, and displays 518.

### ADDITIONAL EMBODIMENTS

Disclosed herein is a system comprising a processor and a non-transitory computer readable medium comprising instructions that, when executed by a processor, cause the processor to: a) obtain or have obtained one or more images; b) determine one or more compositions of a plurality of regions within the one or more images; c) for each region in the plurality of regions, determine a distance between the composition of the region and one or more target vectors; and d) select a candidate region from the plurality of regions, the candidate region having a minimum distance between the composition of the region and one or more target composition vectors in comparison to determined distances of other regions in the plurality. In various embodiments, the non-transitory computer readable medium further comprises instructions that, when executed by the processor, cause the processor to select a portion of the image corresponding to the selected candidate region. In various embodiments, the instructions that cause the processor to determine compositions of the plurality of regions within the image further comprises instructions that, when executed by the processor, cause the processor to deploy a machine learning model.

In various embodiments, the machine learning model performs image classification on the one or more images. In various embodiments, the machine learning model performs image classification one or more images using one or more of a convolutional neural network algorithm, logistic regression algorithm, k-nearest neighbor algorithm, support vector machine algorithm, decision tree algorithm, and random forest algorithm. In various embodiments, the instructions that cause the processor to deploy the machine learning model further comprises instructions that, when executed by the processor, cause the processor to classify the one or more images for a plurality of region classes. In various embodiments, the plurality of region classes comprises at least two, at least three, at least four, or at least five region classes. In various embodiments, the instructions that cause the processor to deploy the machine learning model further comprises instructions that, when executed by the processor, cause the processor to generate target composition vectors of one or more regions of an image.

In various embodiments, the target composition vector is a vector summarizing the classifications of the plurality of region classes for a region. In various embodiments, the instructions that cause the processor to determine a distance between the composition of the region and the one or more target composition vectors further comprises instructions that, when executed by the processor, cause the processor to determine the distance between the region composition vector of the candidate region and the one or more target composition vectors. In various embodiments, the instructions that cause the processor to determine the distance further comprises instructions that, when executed by the processor, cause the processor to determine a cosine distance.

In various embodiments, each of the one or more target composition vectors indicate target percentages of a plurality of region classes. In various embodiments, the non-transitory computer readable medium further comprises instructions that, when executed by the processor, cause the processor to generate a ranked list of candidate regions comprising the selected candidate region and one or more additional candidate regions. In various embodiments, the instructions that cause the processor to generate the ranked list of candidate regions further comprises instructions that, when executed by the processor, cause the processor to rank the candidate region and one or more additional candidate regions according to their corresponding determined distances. In various embodiments, the ranked list of candidate regions ranks the candidate region and one or more additional candidate regions in ascending order of cosine distances. In various embodiments, the one or more images comprise one or more images of a tissue.

Disclosed herein is a method for selecting one or more portions of a tissue, the method comprising: a) obtaining or having obtained one or more images of the tissue; b) determining one or more tissue compositions of a plurality of regions within the one or more images; c) for each region in the plurality of regions, determining a distance between the tissue composition of the region and one or more target composition vectors; and d) selecting a candidate region from the plurality of regions, the candidate region having a minimum distance between the tissue composition of the region and one or more target composition vectors in comparison to determined distances of other regions in the plurality.

In various embodiments, methods disclosed herein further comprise isolating the selected one or more portions of the tissue; and processing the selected one or more portions of the tissue to generate a tissue microarray. In various embodiments, isolating the selected one or more portions comprises sampling one or more tissue cores. In various embodiments, processing the selected one or more portions to generate a tissue microarray comprises: generating a paraffin embedding of the one or more selected portions; sectioning the paraffin embedding of the one or more selected portions; and performing tissue staining of the sectioned paraffin embeddings.

Disclosed herein is a method for selecting one or more portions of a tissue, the method comprising: a) obtaining or having obtained one or more images of the tissue; b) determining one or more tissue compositions of a plurality of regions within the one or more images; c) for each region in the plurality of regions, determining a distance between the tissue composition of the region and one or more target composition vectors; and d) selecting a candidate region from the plurality of regions, the candidate region having a minimum distance between the tissue composition of the region and one or more target composition vectors in comparison to determined distances of other regions in the plurality.

In various embodiments, methods disclosed herein further comprise selecting a portion of the tissue corresponding to the selected candidate region. In various embodiments, methods disclosed herein further comprise isolating the selected one or more portions of the tissue; and processing the selected one or more portions of the tissue to generate a tissue microarray. In various embodiments, isolating the selected one or more portions comprises sampling one or more tissue cores. In various embodiments, sampling one or more tissue cores comprises sampling one or more cylindrical tissue cores.

In various embodiments, processing the selected one or more portions to generate a tissue microarray comprises: generating a paraffin embedding of the one or more selected portions; sectioning the paraffin embedding of the one or more selected portions; and performing tissue staining of the sectioned paraffin embeddings. In various embodiments, obtaining or having obtained the one or more images comprises: performing staining of one or more slides comprising sections from the tissue; and capturing the one or more images of the one or more slides to obtain the one or more images. In various embodiments, the one or more images comprise one or more hematoxylin and eosin (H&E) stained images.

In various embodiments, determining tissue compositions of the plurality of regions within the image comprises deploying a machine learning model. In various embodiments, the machine learning model performs image classification on the one or more images. In various embodiments, the machine learning model performs image classification one or more images using one or more of a convolutional neural network algorithm, logistic regression algorithm, k-nearest neighbor algorithm, support vector machine algorithm, decision tree algorithm, and random forest algorithm. In various embodiments, deploying the machine learning model comprises classifying the one or more images for a plurality of tissue classes. In various embodiments, the plurality of tissue classes comprises at least two, at least three, at least four, or at least five tissue classes. In various embodiments, the plurality of classes comprises two or more of tumor class, stroma class, immune class, necrosis class, or other tissue class. In various embodiments, the plurality of classes comprises each of tumor, stroma, immune, necrosis, or other tissue.

In various embodiments, deploying the machine learning model comprises generating tissue composition vectors of one or more regions of an image. In various embodiments, the tissue composition vector is a vector summarizing the classifications of the plurality of tissue classes for a region. In various embodiments, the tissue composition vector is a 5-element vector summarizing the classifications of tumor, stroma, immune, necrosis, and other tissue for the region. In various embodiments, determining a distance between the tissue composition of the region and the one or more target composition vectors comprises determining the distance between the tissue composition vector of the candidate region and the one or more target composition vectors. In various embodiments, the distance comprises determining a cosine distance. In various embodiments, each of the one or more target composition vectors indicate target percentages of a plurality of tissue classes.

In various embodiments, at least one of the target composition vectors indicates a 100% tumor class. In various embodiments, the at least one of the target composition vectors is expressed as [1,0,0,0,0], wherein the first entry of the vector corresponds to a tumor tissue class. In various embodiments, at least one of the target composition vectors indicates a 100% stroma class. In various embodiments, wherein the at least one of the target composition vectors is expressed as [0,1,0,0,0], wherein the second entry of the vector corresponds to a stroma tissue class. In various embodiments, at least one of the target composition vectors indicates a 50% tumor class and a 50% stroma class. In various embodiments, the at least one of the target composition vectors is expressed as [0.5,0.5,0,0,0], wherein the second entry of the vector corresponds to a stroma tissue class. In various embodiments, a first target composition vector indicates a 100% tumor class, a second target composition vector indicates a 100% stroma class, and a third target composition vector indicates a 50% tumor class and a 50% stroma class.

In various embodiments, methods disclosed herein further comprise generating a ranked list of candidate regions comprising the selected candidate region and one or more additional candidate regions. In various embodiments, generating the ranked list of candidate regions comprises ranking the candidate region and one or more additional candidate regions according to their corresponding determined distances. In various embodiments, the ranked list of candidate regions ranks the candidate region and one or more additional candidate regions in ascending order of cosine distances. In various embodiments, generating the ranked list of candidate regions comprises identifying the one or more additional candidate regions for one or more of a tumor class, stroma class, immune class, necrosis class, other tissue class, tumor-stroma class, or tumor-immune class. In various embodiments, methods disclosed herein further comprise comprising selecting a portion of the tissue corresponding to a candidate region in the ranked list for a tumor tissue class and selecting a portion of the tissue corresponding to a candidate region in the ranked list for a stroma tissue class. In various embodiments, methods disclosed herein further comprise selecting a portion of the tissue corresponding to a candidate region in the ranked list for a tumor-stroma tissue class and selecting a portion of the tissue corresponding to a candidate region in the ranked list for a tumor-immune tissue class.

In various embodiments, methods disclosed herein further comprise selecting five or more candidate regions from the ranked list for a tumor tissue class; selecting five or more candidate regions from the ranked list for a stroma tissue class; selecting five or more candidate regions from the ranked list for a tumor-stroma class; and selecting five or more candidate regions from the ranked list for a tumor-immune class. In various embodiments, the one or more images are captured from the tissue. In various embodiments, the tissue is derived from the subject.

Disclosed herein is a non-transitory computer readable medium comprising instructions that, when executed by a processor, cause the processor to perform the method of any methods disclosed herein. Disclosed herein is a system comprising: a processor; and a non-transitory computer readable medium comprising instructions that, when executed by a processor, cause the processor to perform the method of any methods disclosed herein.

### EXAMPLES

Below are examples of specific embodiments. The examples are offered for illustrative purposes only and are not intended to limit the scope. Efforts have been made to ensure accuracy with respect to numbers used, but some experimental error and deviation should be allowed for.

### Example 1—Example Workflow of Precision TMA Construction

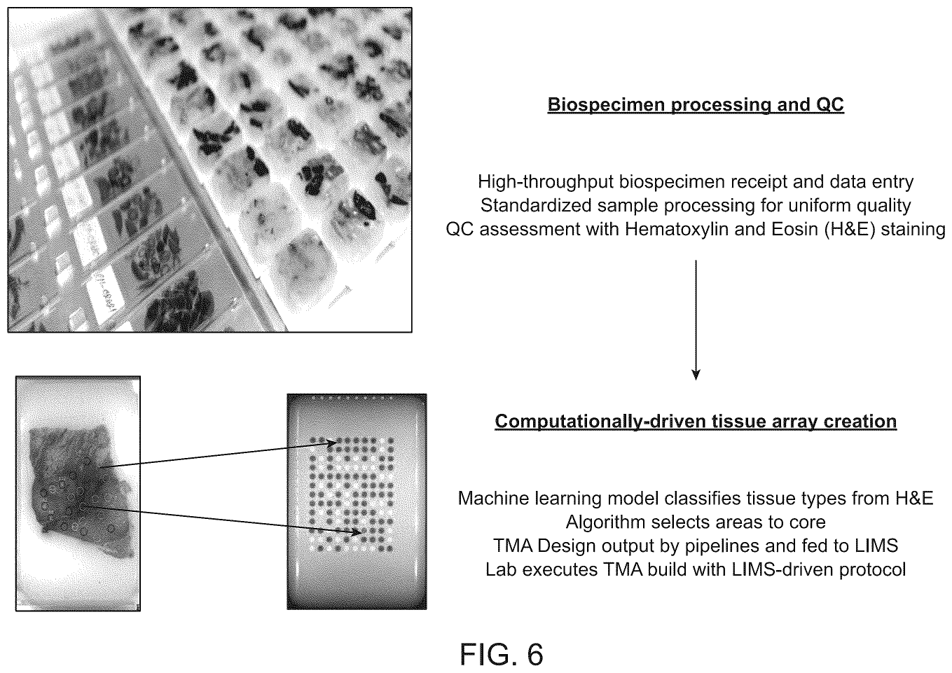

FIG. 6 is an example experimental outline wherein sample tissue was prepared for computational processing that allowed for precision TMA construction. In brief, the top image depicts a representative image of pre-stained and stained tissue images. The accompanying text, on the right, outlines the process by which the biospecimens were processed and quality control (QC) was determined. The first step of this process included creating tissue sections from Formalin-Fixed Paraffin Embedded tissue sample. Next, the sectioned tissue samples were stained with Hematoxylin and Eosin (H&E) and scanned with a digital microscope. Attention is now directed to the bottom image and the accompanying text of FIG. 6. The left-bottom image represents an H&E stained imaged where cores of interest were computationally identified and selected for TMA preparation. These images were subsequently processed through a machine learning algorithm to classify the tissue into tumor, stroma, necrosis, non-tissue, and other tissue classes. Following this tissue classification, the core selection algorithm selected regions of the block to core. After this algorithm was used to select regions of the block to core, automated instructions were utilized to create a TMA on the 3DHistech TMA Grand Master machine (GTMA). Finally, TMAs created on the GTMA were melted and sectioned onto slides.

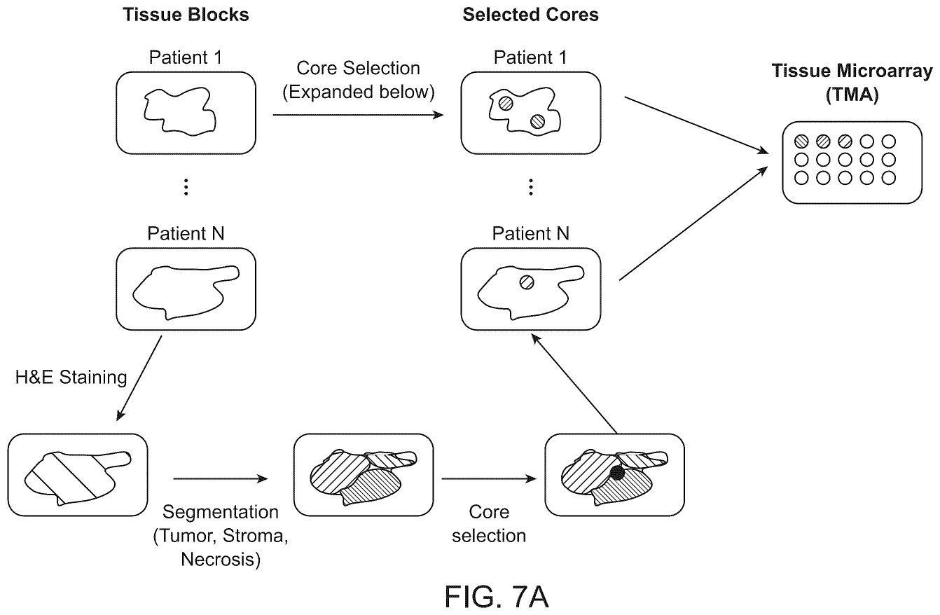

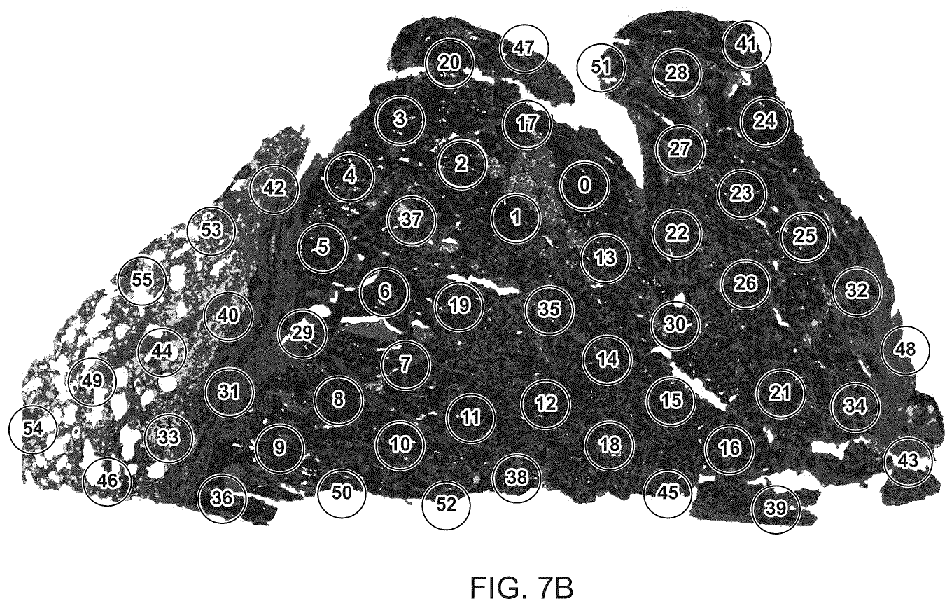

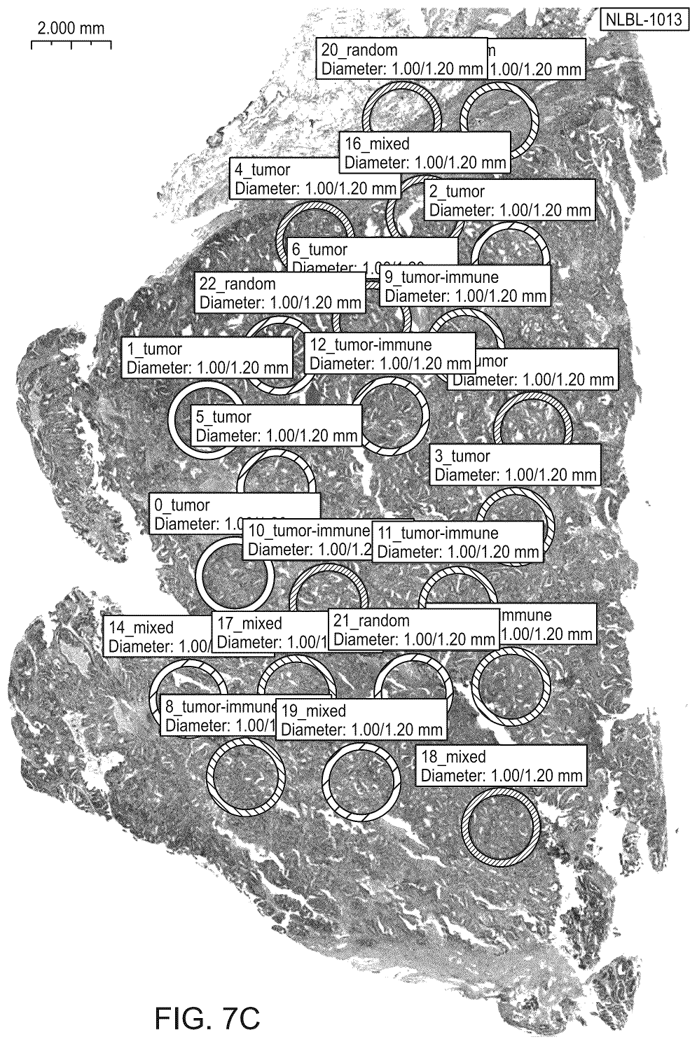

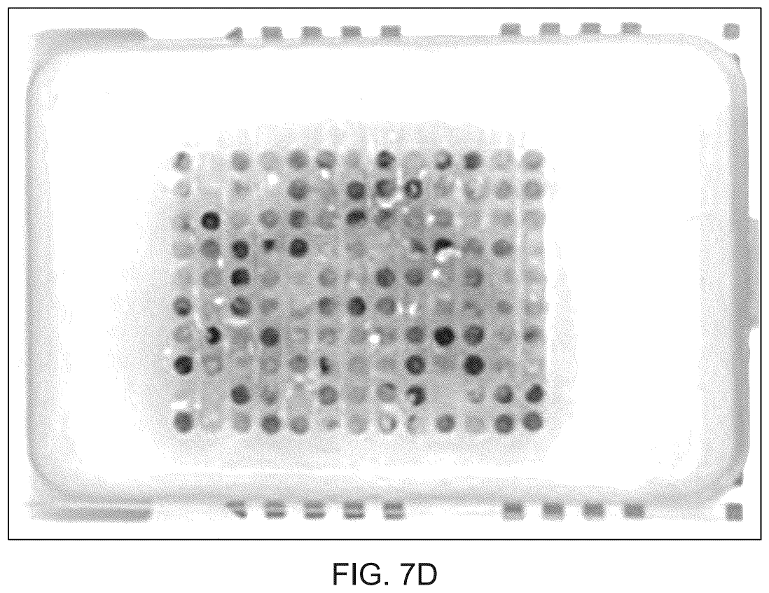

### Example 2—Example Experimental Workflow and Representative Results (FIG. 7A-7D)

FIG. 7A is a representative image of an example experimental and computational workflow used to select cores from patient tissues. In this example, the workflow outlines the process by which patient samples undergo core selection. Briefly, patient tissue blocks underwent H&E staining, segmentation based on tissue classes, and core selection. Cores were then selected from each patient tissue block based on core similarity to reference tissue class vectors. These cores were then added to a TMA.

A representative image of the results of the region segmentation process is presented in FIG. 7B. Region segmentation in this example was facilitated through the following process. Images were processed and were analyzed through a series of automated cloud pipelines built using the open source NextFlow software. These pipelines converted H&E images to ome.zarr and ome.tiff formats, scanned barcode images to identify the slides, and then ran the images through a machine learning model which classified small regions of tissue into tumor, stroma, immune, necrosis, non-tissue, and other tissue. Following the machine learning classification of image regions, the core selection algorithm selected regions of the block to be cored. The algorithm functioned by optimizing the cosine distance of a core's composition to one of three optimal composition vectors in the space [tumor, stroma, immune, necrosis, other]: [1, 0, 0, 0, 0] (100% tumor), [0, 1, 0, 0, 0] (100% stroma), or [0.5, 0.5, 0, 0, 0] (50% tumor and 50% stroma). These vectors were chosen to target the core compositions that were intended to be studied study in this example.

The algorithm calculated the tissue composition of every possible core in the H&E image. Each core was a circular region of a set diameter in the H&E image, and its composition in tissue types was calculated by summing the types of each small region in the image within the given circle. The calculation for all possible cores gave a listing of cores with (x, y) position on the image as well as composition, in the form of a 5-element vector specifying the fraction of the core of each type: [tumor, stroma, immune, necrosis, other].

After calculating the composition for every possible core, the algorithm identified the single core region that had the highest composition of tumor, calculated as the lowest cosine distance to the composition vector [1, 0, 0, 0, 0] (100% tumor).

After this initialization, the algorithm began an iterative process. During each iteration, the algorithm found the best possible next core that was within a minimum set distance of the existing chosen cores. It calculated “best” by optimizing the cosine distance of a core's composition to one of three optimal composition vectors in the space [tumor, stroma, immune, necrosis, other]: [1, 0, 0, 0, 0] (100% tumor), [0, 1, 0, 0, 0] (100% stroma), or [0.5, 0.5, 0, 0, 0] (50% tumor and 50% stroma). Once it found the next best core, it added this core to the list of selected cores, and then began the iterative cycle again to find the next core. The algorithm repeated this process until there were no possible remaining cores on the tissue regions in the image. As shown in FIG. 7B, the circles show cores selected by the algorithm, numbered in the order they were selected. Core 0 was the first core chosen, then core 1, and so on. Core 0 had the highest tumor composition of all possible cores, and subsequent cores optimized for tissue composition as well as core placement for optimal packing.

Attention is now directed towards Table 1, where exemplary tissue composition vectors are displayed in rank order based on similarity to the target vector [1,0,0,0,0] (100% tumor). Based on the similarity to target composition vector (100% tumor), core 1 (50.904% tumor) features the highest similarity and core 5 (38.487% tumor) features the lowest similarity.

TABLE 1
Exemplary tissue composition vectors from a tissue sample.

| Core # | % Tumor | % Stroma | % Immune | % Necrosis | % Other |
|---|---|---|---|---|---|
| 1 | 50.904% | 6.0634% | 8.3501% | 4.2725% | 29.838% |
| 2 | 50.753% | 6.4903% | 14.392% | 4.2287% | 24.102% |
| 3 | 43.403% | 5.8114% | 14.687% | 1.3508% | 34.318% |
| 4 | 43.197% | 12.168% | 11.419% | 0.6932% | 32.171% |
| 5 | 38.487% | 11.761% | 15.556% | 8.7784% | 25.370% |

Attention is now directed towards FIG. 7C, which shows a representative image of selected cores that cover the entire block. The aforementioned algorithm selected a set of cores to be included into 3 TMAs. The goal of spreading patient samples across multiple TMAs was for subsequent processing of sections from these TMAs for separate staining batches in downstream imaging applications, which enables computational analysis to control for staining batch when analyzing patient sample images. To spread cores across TMAs in this way, the algorithm selected a set of 6 tumor cores, a set of 6 stroma cores, a set of 6 tumor-immune cores, a set of 6 tumor-stroma cores, and a set of 3 random cores, and distributed ⅓ of each of these sets randomly to each of the 3 TMAs. A representative image of a completed TMA block through this method is displayed in FIG. 7D.

### Example 3—Validation of Machine Learning with Ground Truth Data

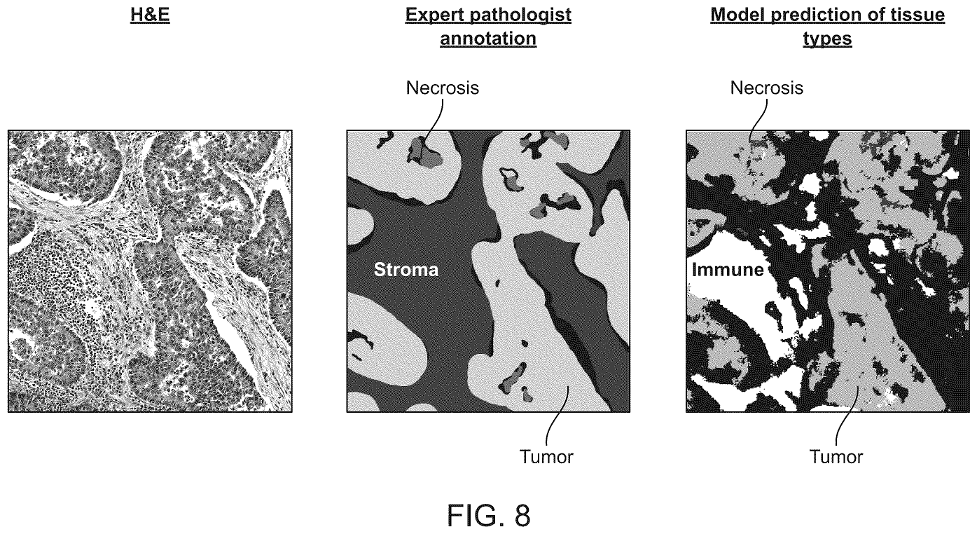

FIG. 8 is a representative image that demonstrates the validation of the machine-learning method in tissue class segmentation of a histological image. The aforementioned machine-learning method was trained on 151 H&E stained whole-tissue slides that were expert-annotated for tissue class segmentation, as described in Amgad et al., Structured crowdsourcing enables convolutional segmentation of histology images, Bioinformatics, Volume 35, Issue 18, September 2019, Pages 3461-3467, which is incorporated by reference in its entirety. Briefly, the machine-learning method achieved tissue class segmentation (tumor, stroma, inflammatory infiltrates, necrosis, and other) through a pre-trained fully convolutional neural network (Long et al., 2015, Proceedings of the IEEE Conference on Computer Vision and Pattern Recognition, pp. 3431-3440, which is incorporated by reference in its entirety). Data augmentation, shifting and cropping of annotated images, was implemented to improve model robustness and generalizability. Model optimization was achieved using an adaptive moment estimation (Adam) optimizer, e.g. in lieu of classical stochastic gradient decent. A cross-entropy loss function was implemented to account for and minimize class imbalance. Representative results of the aforementioned machine learning model are as follows. The left panel features a H&E stained histological image. This image was annotated by an expert pathologist for stroma, tumor, necrosis, and non-tissue tissue classes (middle panel) for the purposes of generating expert-labeled ground truth data to evaluate the accuracy of the machine learning model. The right panel of FIG. 8 shows the tissue class segmentation results of the aforementioned machine learning method when set to evaluate the H&E stained image in the left panel of FIG. 8. The machine-learning method was able to identify immune regions which were not identified by the pathologist. For the other regions of stroma, tumor, necrosis, and non-tissue classes (which were identified by the pathologist) the machine learning method achieved strong alignment with the expert annotated H&E stained image, demonstrating the tractability of this approach and its accuracy.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A system comprising:
a processor; and
a non-transitory computer readable medium comprising instructions that, when executed by a processor, cause the processor to:
a) obtain or have obtained one or more images;
b) determine one or more compositions of a plurality of regions within the one or more images;
c) for each region in the plurality of regions, determine a distance between the composition of the region and one or more target vectors; and
d) select a candidate region from the plurality of regions, the candidate region having a minimum distance between the composition of the region and one or more target composition vectors in comparison to determined distances of other regions in the plurality.

2. The system of claim 1, wherein the non-transitory computer readable medium further comprises instructions that, when executed by the processor, cause the processor to select a portion of the image corresponding to the selected candidate region.

3. The system of claim 1, wherein the instructions that cause the processor to determine compositions of the plurality of regions within the image further comprises instructions that, when executed by the processor, cause the processor to deploy a machine learning model.

4. The system of claim 3, wherein the machine learning model performs image classification on the one or more images.

5. The system of claim 4, wherein the machine learning model performs image classification one or more images using one or more of a convolutional neural network algorithm, logistic regression algorithm, k-nearest neighbor algorithm, support vector machine algorithm, decision tree algorithm, and random forest algorithm.

6. The system of claim 3, wherein the instructions that cause the processor to deploy the machine learning model further comprises instructions that, when executed by the processor, cause the processor to classify the one or more images for a plurality of region classes.

7. The system of claim 6, wherein the plurality of region classes comprises at least two, at least three, at least four, or at least five region classes.

8. The system of claim 3, wherein the instructions that cause the processor to deploy the machine learning model further comprises instructions that, when executed by the processor, cause the processor to generate target composition vectors of one or more regions of an image.

9. The system of claim 8, wherein the target composition vector is a vector summarizing the classifications of the plurality of region classes for a region.

10. The system of claim 8, wherein the instructions that cause the processor to determine a distance between the composition of the region and the one or more target composition vectors further comprises instructions that, when executed by the processor, cause the processor to determine the distance between the region composition vector of the candidate region and the one or more target composition vectors.

11. The system of claim 10, wherein the instructions that cause the processor to determine the distance further comprises instructions that, when executed by the processor, cause the processor to determine a cosine distance.

12. The system of claim 1, wherein each of the one or more target composition vectors indicate target percentages of a plurality of region classes.

13. The system of claim 1, wherein the non-transitory computer readable medium further comprises instructions that, when executed by the processor, cause the processor to generate a ranked list of candidate regions comprising the selected candidate region and one or more additional candidate regions.

14. The system of claim 13, wherein the instructions that cause the processor to generate the ranked list of candidate regions further comprises instructions that, when executed by the processor, cause the processor to rank the candidate region and one or more additional candidate regions according to their corresponding determined distances.

15. The system of claim 14, wherein the ranked list of candidate regions ranks the candidate region and one or more additional candidate regions in ascending order of cosine distances.

16. The system of claim 1, wherein the one or more images comprise one or more images of a tissue.

17. A method for selecting one or more portions of a tissue, the method comprising:
a) obtaining or having obtained one or more images of the tissue;
b) determining one or more tissue compositions of a plurality of regions within the one or more images;
c) for each region in the plurality of regions, determining a distance between the tissue composition of the region and one or more target composition vectors; and
d) selecting a candidate region from the plurality of regions, the candidate region having a minimum distance between the tissue composition of the region and one or more target composition vectors in comparison to determined distances of other regions in the plurality.

18. The method of claim 17, further comprising:
isolating the selected one or more portions of the tissue; and
processing the selected one or more portions of the tissue to generate a tissue microarray.

19. The method of claim 18, wherein isolating the selected one or more portions comprises sampling one or more tissue cores.

20. The method of claim 18, wherein processing the selected one or more portions to generate a tissue microarray comprises:
generating a paraffin embedding of the one or more selected portions;
sectioning the paraffin embedding of the one or more selected portions; and
performing tissue staining of the sectioned paraffin embeddings.
